(19) United States
(12) Patent Application Publication
(10) Pub. No.: US 2022/0023482 A1
(43) Pub. Date: Jan. 27, 2022

(54) PATHOGEN DECONTAMINATION FOR INDOOR ENVIRONMENTS USING ULTRAVIOLET RADIATION

(71) Applicant: Evergreen UV, LLC, Memphis, TN (US)

(72) Inventor: David Robert Skelton, Memphis, TN (US)

(21) Appl. No.: 17/386,498

(22) Filed: Jul. 27, 2021

Related U.S. Application Data
(60) Provisional application No. 63/057,012, filed on Jul. 27, 2020.

(51) Int. Cl. A61L 9/20; A61L 2/10

(52) U.S. Cl. CPC A61L 9/20; A61L 2/10; A61L 2202/122; A61L 2209/16; A61L 2202/11; A61L 2209/12

(57) ABSTRACT

Decontamination systems and tools for inactivating pathogens in an indoor environment using ultraviolet radiation are disclosed. Systems and tools for inactivating pathogens include one or more UVC light sources and a UVC control module for controlling the one or more light sources. The UVC control module may receive inputs with data describing aspects of the indoor environment or operating parameters of an HVAC system associated with the decontamination system.

### CROSS-REFERENCE TO RELATED APPLICATIONS

This application claims the benefit of priority to and incorporates by reference the entire disclosure of U.S. Provisional Patent Application No. 63/057,012 filed on Jul. 27, 2020.

### FIELD OF THE INVENTION

Various embodiments of the present invention generally relate to processes, systems, and techniques for inactivating pathogens in an indoor environment using ultraviolet radiation. In particular embodiments of the invention, tools and devices are provided for decontaminating air and surfaces within heating systems, ventilation systems, air conditioning systems, and other similar systems servicing an indoor environment by destroying airborne pathogens circulating through such systems, and/or biological contamination of surfaces within such systems, with ultraviolet radiation.

### SUMMARY

Promoting safety is an important aspect of pursuits in business, education, culture, recreation, or any other endeavor that involves people interacting or performing activities within indoor environments. Many different types of buildings, manufacturing plants, commercial offices, educational settings, medical centers and offices, recreational centers, and other indoor facilities that have heating systems, ventilation systems, air conditioning systems, and other similar systems, are susceptible to pathogens circulating through the HVAC (heating, venting, air circulating) systems of such places. Such pathogens might be viruses, bacteria, spores, or other harmful allergens, microorganisms or substances that can negatively impact the health of people contained within the indoor environment.

Enhanced tools and techniques are needed that can alleviate the problems associated with pathogens contaminating surfaces and/or circulating within different indoor environments, to reduce sickness and absenteeism, to reduce pathogen load, and to improve indoor air quality (IAQ). Other benefits and advantages can be realized in reducing HVAC energy costs and improving air handling unit (AHU) thermal efficiency, for example. Also, technological improvements are needed to reduce labor and maintenance costs, while also reducing dependency on potentially hazardous and environmentally unfriendly chemical-based cleaning products.

According to aspects of the disclosure, a decontamination system includes an HVAC duct, a UVC light source disposed within the HVAC duct, and a UVC control module comprising a processor and memory. The UVC control module in in electrical communication with the UVC light source and is configured to control operation of the UVC light source.

According to aspects of the disclosure, a decontamination system includes a frame configured to be attached to an HVAC vent, and at least one UVC light secured to an interior of the frame. At least one wall of the frame includes a plurality of collimator blades that permit air to pass from an interior of the frame to an exterior of the frame.

According to aspects of the disclosure, a decontamination wand includes a housing, a battery disposed within the housing, a UVC light source selected from the group consisting of a low pressure mercury lamp and a UVC light source, a spring-loaded safety trigger that is biased in an off position, and a shield disposed around at least a portion of the UVC light source, the shield comprising a UVC reflective material on an portion of the shield facing the UVC light source.

### BRIEF DESCRIPTION OF THE DRAWINGS

A more complete understanding of embodiments of the present invention may be obtained by reference to the following Detailed Description when taken in conjunction with the accompanying Drawings wherein:

FIG. 1 is a schematic of a decontamination system according to aspects of the disclosure;

FIG. 2 is a schematic of a combined active and passive decontamination system according to aspects of the disclosure; and

FIGS. 3A and 3B are side and front views, respectively, of a UVC wand according to aspects of the disclosure.

### DETAILED DESCRIPTION

In connection with describing various embodiments of the invention herein, reference may be made to Ultraviolet Germicidal Irradiation Handbook—UGVI for Air and Surface Disinfection (“Handbook”—Wladyslaw Kowalski, Springer-Verlag Berlin Heidelberg 2009, ISBN 978-3-642-01998-2), the entire disclosure of which is incorporated herein by reference. Except where noted otherwise, references to Tables, Figures, or Chapters herein are connected to the Handbook. Also, other external sources of research or information mentioned in the Handbook may be referenced herein.

It can be appreciated that various embodiments of the invention described herein, while illustrated by way of example in the context of certain environments such as the interior of buildings, can be extended to other applications or implementations. For example, embodiments of the UVC systems described here may be installed in many different types of indoor environments, vehicles (e.g., cars, trucks, ships, boats, buses, etc.), buildings, structures, or other interior environments which employ HVAC systems, ductwork for ventilation, or generally implement systems which circulate air through the interior environment.

In developing various embodiments of the invention, the inventors have leveraged the scientifically proven germicidal properties of UVC light (e.g., 200 nm-280 nm wavelength). UVC light has helped to eradicate measles and tuberculosis outbreaks. It is effective to some degree on all pathogens which contain DNA or RNA. For example, UVC light is particularly effective for inactivating the corona virus (COVID-19) and also works well on the seasonal flu and H1N1 flu strain. It is also effective on mold and Legionella bacteria among many other pathogens. Unlike medications, pathogens do not build up immunity over time or mutate to form resistance to UVC light. One can think of UVC as nature's sterilizer as all sunlight contains some wavelengths in this spectrum of light.

All UVC light is not the same. For example, 265 nm wavelength UV light has been scientifically proven to have the highest germicidal effectiveness. Low pressure mercury bulbs primarily emit 253.7 nm UVC light, which is sufficiently close to 265 nm to provide adequate germicidal effectiveness as well. LED UVC light can be “tuned” to UV wavelengths that are ideal for inactivation of virus and bacteria microbes. It is also important that the intensity of the UVC light be sufficient to perform the desired germicidal effect. Inactivation rates are determined primarily by the intensity of the lamp and the exposure time (dwell time). The UVC light that many in the industry use for condenser coil cleaning or claim to reduce pathogens often does not provide the adequate germicidal intensity. There are scientifically determined and published UVC K values for almost all known pathogens. K-value inactivation intensity is typically measured in Joules, or microwatts (intensity) generated per second (dwell time). In installations near a cooling coil, airborne pathogens traveling at 500 f/m (average) only achieve 0.25 to 0.5 seconds of exposure time. Accordingly, any installed in-line UVC system should provide the required intensity during the period time the air is exposed to the UVC which may be less than one second, for example, in certain applications.

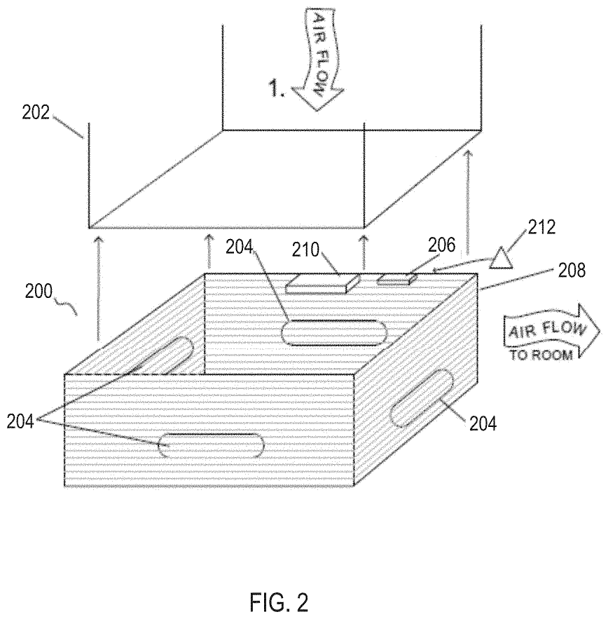

The germicidal effectiveness of UVC is illustrated in FIG. 2.1 of the Handbook, where it can be observed that germicidal efficiency reaches a peak at about 260 nm-265 nm. This corresponds to the peak of UV absorption by bacterial DNA (Harm 1980). The germicidal effectiveness of UVC and UVB wavelengths can vary between species. Low pressure mercury vapor lamps radiate about 95% of their energy at a wavelength of 253.7 nm, which is sufficiently close to the DNA absorption peak (260 nm-265 nm) to provide relatively high germicidal effectiveness (IESNA 2000).

Von Sonntag (1986) reports that DNA has a peak of UV absorption not only at 265 nm but also at 200 nm. Most of the absorption at 200 nm occurs in the DNA backbone molecules of ribose and phosphate. At 265 nm, most of the absorption occurs at the nucleotide bases, thymine and adenine, and cytosine and guanine, but dimers of thymine are by far the most common UV photoproducts. In RNA-based microbes, uracil is also involved in UV absorption in place of thymine but not necessarily to the same degree. FIG. 2.9 of the Handbook compares the absorption spectrum of uracil with that of thymine. It can be observed that not only are the absorption spectra very similar for these nucleotides, but that the mercury emission line at 254 nm is more nearly aligned with the peak absorption of uracil.

Mathematical modeling of the UV disinfection process provides a basis for sizing ultraviolet disinfection equipment and for interpreting test results. It also allows for adaptation of UV systems to specific disinfection processes and for the disinfection of specific microorganisms. When the UV dose results in a 90% disinfection rate (10% survival), it is known as a D90. The D90 value is commonly used as an indicator of system size and can be used to assess the survival rate of individual microbes. Also common is the D99, or the dose that results in 99% inactivation.

The effect of relative humidity (RH) on virus UV susceptibility is much less pronounced than it is in bacteria. Table 4.7 of the Handbook summarizes all the available data on RH effects for viruses and provides a ratio for dry to wet conditions. The UV lamp is usually the main component of any disinfection system and must meet certain standards and performance criteria, and a number of guidelines and standards are currently available (NFPA 2008, ANSI 2005a, b, UL 2004, IESNA 2000). See Chapter 11 of the Handbook for a review of the applicable electrical and mechanical guidelines and standards for UV lamp applications. The lamp ballast must also be designed to meet certain standards, as must lamp fixtures and associated electrical wiring.

With reference to Chapter 6.10 of the Handbook (“Controls and Integral Sensors”), control units are available that will allow the UV output of lamps to be manually or automatically adjusted. One application is in an operating room of a hospital, for example. Overhead UV systems in which the irradiance levels are dialed down during procedures but turned up during unoccupied periods for room disinfection. Integral UV sensors are also available for monitoring UV output of in-duct systems, enabling verification of performance or indicating when UV lamps may need replacement.

With reference to Chapter 7.5 of the Handbook (“UV System Optimization”), the performance of UVGI systems can be optimized to produce maximum inactivation rates while minimizing energy costs if attention is paid to certain aspects of design. Both reflectivity and duct length, for example, can be used to reduce required UV lamp power. Reflectivity will increase the average irradiance while duct length will increase the average UV dose (by increasing the exposure time). Ignoring, for the moment, fan power considerations, there are design approaches that can increase the performance from a disinfection point of view. There are several variables that define the design and operation of any UVGI system, and these include the dimensions (width×height×length), the lamp coordinates (x, y, z), airflow (Q), reflectivity (p), lamp UV wattage (P), lamp length (l), and lamp radius (r). Some of the variables have optimum values that are fairly well known. The optimum air velocity for most lamps is 2.54 m/s (500 fpm) while the typical operating velocity range of most HVAC systems is about 2-3 m/s (400 fpm-600 fpm). The optimum operating air temperature is considered to be 20-22° C. (68-72° F.). The critical design parameters of UVGI systems and their impact on performance has been evaluated using dimensional analysis (Kowalski et al. 2003, 2005). There are eight dimensionless parameters, excluding RH, that define UVGI system performance, and these are defined in Table 7.1 of the Handbook.

FIG. 7.11 of the Handbook compares the effects of specular reflectivity and specific dose (Kowalski et al. 2005). Increasing reflectivity produces an approximately linear increase in inactivation rates but that gains level off as inactivation rates near 100%. It can be seen from this chart that if the same inactivation rates can be achieved by increasing reflectivity, then the expense of increasing lamp power may be unnecessary, but this is ultimately a matter of economics (e.g., lamp power vs. materials). Economic optimization of UVGI systems is possible by assigning a cost to any system in terms of the defining parameters. The critical cost determinants include the UV lamp power, the duct length, and the reflectivity. However, the operating cost of a UV system may be dwarfed by the fan power consumption if the pressure losses are high, as they might be if a filter is added, or if light baffles are used.

In developing advancements in the technology of UVC fixtures, UVC energy has been proven to inactivate and destroy microbial pathogens. In fact, there are no known RNA/DNA-based pathogens that are resistant to a proper measured dose of UVC energy.

With regard to surface disinfection in HVAC systems, UVC fixtures can be placed proximate to HVAC cooling coils to inactivate pathogens and biofilm that pass through, cling to, and insulate, cooling coil fins. Such systems are referenced as surface treatment or coil disinfection systems. Because UVC energy is cumulative, and because such coil cleaning systems generally treat coil surfaces 24/7, only low doses (intensity) of UVC energy is required to disinfect biofilm and growth on coil fins. Low-dose coil cleaning UVC systems remove biofilm that insulates coil surfaces to restore coils to as-new/as-spec, reduce static pressure, improve air flow, restore heat exchange, and ultimately reduce work on the fan to reduce amperage-draw and therefor reduce energy consumption. Coil cleaning results in energy savings.

With regard to air disinfection in HVAC systems (IAQ systems), UVC fixtures may be placed into the ventilation ductwork to inactivate airborne pathogens as they travel through the duct system. Such a system is commonly referenced as an air disinfection or IAQ system. A current practice is to inject UVC lamps into ductwork to treat (disinfect) airborne pathogens that travel through HVAC systems, as air is mechanically moved (fan-driven) from one or more return vents, through one or more ventilation ducts, into a single, common plenum where it is conditioned (heated/cooled) and circulated back into the space through a supply vent(s).

As all facility air must pass through such coils to be heated or cooled, placement of UVC systems in the air plenum and proximate to the cooling/heating coils are common for both IAQ and coil cleaning systems. As a general rule, UVC energy exposure is cumulative. If treatment of a contaminated coil surface required 2000 microwatts of UVC energy, one could generate only 20 microwatts of UVC energy for 100 seconds, 200 microwatts for 10 seconds, or 2000 microwatts for only one second of treatment with similar disinfection results. Although some microbial reactivation may occur if dosage is too low.

Air moving at 500 fpm (common air speed though ventilation system) through a duct travels at 8.3 feet per second, while the distance from a cooling coil to a distribution fan is usually about 4 feet or less, so the time of treatment (exposure) is often only about ¼ to ½ second of exposure time. Pathogen reduction percentage is a product of intensity of UV energy×exposure time, so high doses of UVC energy can be required to achieve high disinfection rates in moving air when exposure times are short. Current practice is to install either a low-dose coil cleaning system for maintenance reduction and energy efficiency; or to install a high-dose air disinfection system for airborne pathogen reduction and improved IAQ. In fact, UV systems are often referenced as a “coil cleaning” or “air disinfection” systems.

It is also important that in-line UVC system installations consider the environment that they are being asked to treat. Systems must be designed, manufactured and installed with the desired germicidal reduction in mind. Locker rooms are different than classrooms. Gymnasiums may require a different system than lunchrooms, for example. When designed and installed properly, an in-line UVC system can reduce airborne pathogens by >95% on first pass, and reduce the surface pathogen load within the treated space by 50%.

With regard to variable dose systems, an IAQ air disinfection system can require very high dose of UV because of short dwell times. Therefore, IAQ systems are not as energy efficient as coil cleaning systems that require much lower doses of UV. Traditional UV systems are either energized 24/7, or are cycled on and off with fan cycles. An advantage of 24/7 operation is that coils can quickly colonize (by biologicals) when the UV system is off, contaminating and seeding the air within the facility when fan operation resumes. An advantage of cycling UV with fan on/off operation is a savings in energy consumption and extension of lamp life. The purpose of a variable dose UV system is to extend lamp life, reduce energy consumption, and to consistently deliver the ideal dosage of UVC intensity required to achieve desired results (IAQ vs. coil cleaning) as facility conditions change, such as occupied/unoccupied, fan on/off, airborne pathogen detection, high contamination risk (flu season, large gatherings, known threats) and other client pre-sets.

In various embodiments, a variable dose UVC lamp array (e.g., multiple lamps) can be installed proximate to cooling coils. With all lamps energized: maximum airborne pathogen inactivation, maximum IAQ, and minimum energy efficiency. During times of fan-on operation, high doses of UVC energy are required for high levels of airborne pathogen inactivation disinfection as dwell times are only about 0.5 seconds. When only a selected lamp of the array is energized during fan off or low flow periods, when no or minimal air is flowing through the duct, this promotes coil cleaning and maximizes energy efficiency. During fan off periods there is minimal airborne pathogen inactivation and any additional UVC light added to the system is inefficient. During times of fan-off or reduced fan operation (some fans/fan walls are engineered to cycle fan speed from high to low, but is never or rarely completely inactive), only low doses of UVC energy are required for high levels of coil biofilm inactivation as dwell times total 86,400 seconds per day.

In certain embodiments, all lamps can be energized and stay energized, but lamp energy/output is dimmed to conserve energy and reduce UV output to achieve the benefits of IAQ and/or coil disinfection. This is generally not possible with mercury tube lamps, for example, but very achievable with LEDs. A hybrid combination of PLL lamp-on/fan-on setting for air disinfection IAQ, LED UVC (only) during fan-off operation can be implemented to take advantage of the beneficial properties of both technologies.

In various embodiments, variable dose UVC applications can be used to maintain sufficient UVC exposure on the coils to inhibit pathogen growth even when the air handler fan is off. When the air handler's fan is on and circulating air through the building, sensors can be configured to automatically engage the UVC array to illuminate at 100%, or in proportion to the airflow, and at a reduced UVC output during periods of low or no airflow to maintain adequate disinfection of the coils. Power can be applied to alternating UVC lamps or fixtures within the array, or to a different type of fixtures such as strobing LED UVC during times of reduced airflow. The number of UVC lamps in the array to be turned off during down time might never be 100%. Determining the number or selection of UVC lamps to turn off during times of reduced airflow can be determined in a number of ways, including by use of chamber airflow sensors, electrical current detection of the fan itself, or other detected conditions or methods. A benefit to this approach is restricting harmful pathogen growth on coils, drain pans, and in vicinity of UVC array to maintain energy efficiency and health benefits. Also, less energy is consumed compared to an always-on UVC array.

It can be seen that variable-dose systems can achieve multiple objectives. Maximum pathogen and allergen inactivation are provided during occupied use of an indoor environment to improve IAQ and occupant performance by eliminating the pathogens that cause infection, sickness and disease. Minimum energy consumption can be realized by performing only enough work required to meet coil cleaning/IAQ goals. Extended lamp life can be provided as lamps are energized only when required to meet HVAC objectives, and individual lamp life can easily be leveled by alternating and averaging specific lamp on/off time. In certain embodiments, hour counters and/or radiometers can be bundled with variable dose systems to maximize lamp life and energy efficiency, while promoting lamp efficacy to inactivate pathogens. Variable dose UV sources can communicate with, be controlled by, and/or be monitored by building automation systems, cloud-based smart devices, and other remote devices. Such remote systems allow over-rides and programmed pre-sets for unique and/or client-driven needs (e.g., large gatherings, outbreaks of sickness (flu season, exposure to known infectious individual), removal of cooking odors, etc.).

IAQ dosing systems can be employed in conjunction with, or instead of, variable dose systems. UV systems can only provide high levels of airborne pathogen inactivation and reduction during system-on/fan-on operation, but 24/7 operation consumes energy and reduces lamp life. UVC systems controlled by fan-on operation reduces energy consumption, but can cause biological growth and colonization on cooling coils. Bundling UV systems with fan sensors, occupant sensors, and/or usage timers can significantly reduce and improve electrical use. Turning lamps on/off with fan sensors can increase lamp life and energy efficiency, but can accelerate biogrowth on coils during fan-off cycles. For example, cycling lamp arrays on for 15 minutes per hour of fan-off operation can be sufficient to keep coils clean. Turning lamps on/off with fan sensors can increase lamp life and energy efficiency, although fan operation is often controlled by temperature changes: the fan turns on in hotter weather as cooling needs increase, and turns on in winter as heating needs increase. However, there are many times when fans are cycled off for extended periods of time because of stabilized temperature within the facility, or when HVAC system is purposely turned off.

In certain embodiments, on-demand dosing systems use occupant-based IAQ technology to reduce airborne pathogen levels independent of or with less consideration given to HVAC settings. A UVC source might always be activated during fan-on cycles, but also preset to cycle on and off during fan off operation to specifically deodorize and decontaminate air. Cycle times of 15 minutes on and 30 minutes off, for example, may suffice to reduce overall pathogen load on coils and distribute pathogen-free air at night, on weekends, or during other inactive times. For greater efficiency, on-demand dosing could also be energized only when sensors indicate occupancy. System over-rides can be used to allow for occupant-based demand, such as during high occupancy (e.g., parties), higher risks (e.g., flu season or outbreaks), personal preferences, or outlier situations (cooking odor).

With regard to combination mercury/LED type systems, to further reduce energy use of the UVC components, an LED UVC source may be used during reduced airflow periods for coil cleaning, as opposed to alternating mercury UVC lamps for the same effect. An LED UVC source can be set to strobe for additional energy efficiency and longer useful life. Using an LED alternative reduces the energy consumption of the UVC array due to the lower power required to operate. The bulb and LED UVC replacement schedule may also be extended two to three times the normally recommended interval.

With regard to reflective materials which can be used in association with various aspects of the invention, galvanized steel, Teflon, aluminum and brushed aluminum provide suitable UV-reflective surfaces. Also, a material marketed under as POREX Virtek® sintered PTFE can be useful for ultraviolet light reflectivity. With over 97% average reflectance from 250 nm to 400 nm, POREX Virtek PTFE or similar reflectors can be used to maximize disinfection rates and minimize system cost.

In various embodiments of the invention, the inventors have recognized the importance of configuration of the apparatus, devices, and systems described herein. For example, the more UVC photons a system can emit into a given chamber of air and/or the longer the chamber of air is exposed to the UVC photons, the more efficient the system will be. Factors such as bulb wattage, bulb size, bulb position, reflective material, duct length, duct baffle structure, and fan speed, among other factors, are variables that make a difference in performance of the system.

In various embodiments, the placement of the UVC system in an HVAC system (e.g., distance from coil or other key locations) can be critical for promoting energy savings and decontamination reasons. In one embodiment, the most efficient site for UVC system placement is an equal distance from the condenser coils and the fan of the HVAC system. However, with the placement of reflective materials into the system the optimal UVC lamp position may be varied accordingly to promote the most efficient and effective arrangement. The inventors have considered the pros and cons for placing lamps upstream of coil or downstream of coil. Using chill-corrected lamps and proper measured dose, for example, can mitigate the advantages otherwise associated with upstream installations.

With regard to maximizing the UV wavelength exposure to the air flow with fixed versus moving reflective surfaces, the inventors have developed use and placement of non-fixed, moving reflective materials. In one embodiment, slices or strips of highly reflective UVC material, for example, can be placed in the duct or on the UVC lamp in a manner designed for the slices to move and fall between the UVC fixture/bulbs and the HVAC fan when the fan is not running reflecting the UVC light onto the coils thus improving coil cleaning. These slices or strips are then carried by the flowing air to the sides of the duct when the fan is operating, reflecting the UVC light within the “kill chamber” or “kill zone” thus improving airborne pathogen reduction without significantly restricting air flow within the system.

Using reflective materials on plenum walls can increase total measured UV dose. Also, the higher the reflectivity of the material, and the more that the reflective material extends the output of the lamp, then the lower that the actual lamp intensity (wattage) needs to be. Because moving air decontamination requires more UVC energy than fixed surfaces, the focus can be on extending UVC energy down the air path, perhaps even into the fan opening. Also, any increase in total dwell time or total intensity will have a corresponding increase in pathogen inactivation.

In various embodiments, the inventors have discovered the usefulness of UVC LEDs for in-duct air and coil disinfection applications. LEDs can be dimmable (e.g., for variable dose systems), can be strobed (e.g., to extend lamp life), can reduce shortages (e.g., lamp, ballast and socket), can eliminate mercury contamination, can reduce energy consumption, and can realize the many other benefits of using LEDs. As described herein, a unique hybrid system can be developed, using LED systems and PLL lamps, to provide the best of both worlds: high intensity PLL lamps can be used for air disinfection solely when the fan is on, and low energy-use LEDs can be used during fan off periods for coil disinfection. By strobing the LEDs (e.g., on for one minute, off for three minutes), for example, coils can remain substantially free of microbial contamination and the benefits of having clean coils can be achieved. By using LEDs, lamp life can be substantially extended, and energy efficiency can be optimized.

In various embodiments of the invention, the inventors have developed a new standard for UV and UVC systems used in connection with HVAC systems. The UVC system can be installed and sized based on the HVAC system in use, the size of the room or rooms to be conditioned, the airborne pathogen load of the room or rooms based on occupancy, room use, and room location, among relevant attributes of the indoor environment. The UVC system can be designed to achieve the level of airborne pathogen eradication selected by the user, and/or eradication levels can be based on published scientifically accepted K values for known pathogens. The systems can be configured to monitor HVAC operations and fan speed to optimize energy savings and desired airborne pathogen eradication. The active systems may also use computer systems, computing devices, software, algorithms, controllers, or other like components to control the HVAC system to maintain the desired airborne pathogen eradication during HVAC downtime and seasonal changes. Multi-lamp active systems can be provided to systematically control the UVC light array to maximize energy efficiency, reduce maintenance costs, and optimize airborne pathogen eradication.

Various embodiments of the invention provide effective pathogen reduction and control. The calculated scientific and measured dose of UVC germicidal energy installed within HVAC systems can inactivate and eliminate infectious RNA/DNA-based pathogens, viruses, bacteria, or spores, regardless of the specific strain or mutation, regardless of drug resistance, and of the concentration of colony forming units (CFUs). Inactivation and elimination of infectious airborne pathogens has been proven to reduce the occurrence of infectious pathogens on high-touch surfaces (e.g., door knobs, kitchen counters, etc.). Inactivation of airborne and surface pathogens has been proven to reduce the occurrence and severity of infection, sickness and disease. Also, with regard to non-infectious pathogen reduction, benefits can be realized in the form of elimination of odors and odor-causing bacteria, and elimination of mold spores and mold colony-forming units.

With respect to energy savings, UVC light energy when employed by the systems described herein, and when installed proximate to HVAC cooling coils, eliminates the biofilm that insulates coil fins, improves air flow, restores heat exchange efficiency, and reduces fan amperage. The systems can provide HVAC equipment life cycle extension: as workload is reduced, life cycles increase. For example, the combination of UVC combined with a standard MERV 13 pleated filter has the air cleaning equivalence of a comparatively more expensive, air-restricting, and energy-inefficient HEPA filter.

In other aspects of the invention, maintenance reduction and savings can be achieved because the present systems use UVC to clean non-chemically. For example, chemical coil cleaning often uses abrasive and toxic chemicals, requires system shutdown, and is labor intensive. The UVC systems described herein can reduce or eliminate the chemicals and labor required to keep coils clean.

In summarizing certain unique features of the present invention, it will be appreciated that the inventors have developed a variable dose UV system that provides ideal coil cleaning during fan-off conditions, plus ideal air disinfection during fan-on conditions. Other less effective systems have attempted to provide either ideal coil cleaning or ideal air cleaning, but the present systems combine the benefits of both approaches by varying the dosage based on various, predetermined sensor inputs. In various embodiments, the present systems have made effective use of dimmable UVC technology within AHUs wherein variable dose can be accomplished by turning lamps on and off, or by dimming lamps. In certain embodiments, the present systems effectively cycle UVC air disinfection on/off based on occupancy demand. Prior systems are either active all the time (and are not energy efficient), or they cycle on/off with the fan. This type of fan-based cycling is especially not ideal during spring and fall seasons of the year when ambient temperature is similar to internal thermostat settings, meaning the system does not regularly cycle on/off and may not cycle at all. In contrast, embodiments of the present invention can be programmed to turn the fan on/off as a way to disinfect indoor air on a scheduled cycle to promote acceptable IAQ, regardless of automated or thermostatically-controlled HVAC heat/cool cycles.

In other beneficial aspects of the present invention, the present systems can combine high output mercury lamps for high level airborne inactivation, with low-energy LED UVC fixtures for energy-efficient coil cleaning. The present systems can employ LED technology for HVAC systems, including using LEDs for HVAC variable dosing. LEDs are easily dimmable, and/or can be strobed (multiple on/off operation is easily achievable and does not reduce system life). A preferred LED system can alternatively activate all fixtures during fan-on operation, and then strobe the lamps on and off (e.g., on for one minute, off for five, on for one, off for five, etc.) based on preferred coil cleaning requirements.

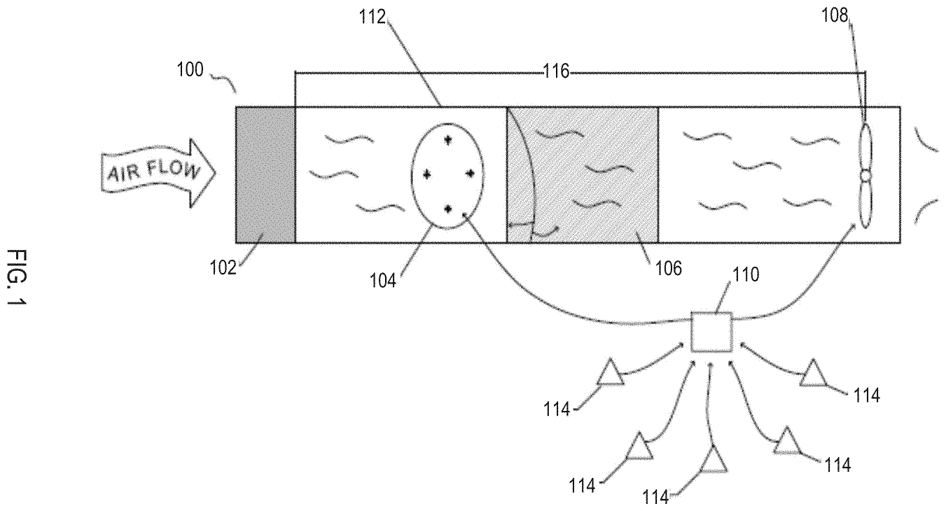

FIG. 1 is a schematic of a decontamination system 100 according to aspects of the disclosure. FIG. 1 illustrates how a decontamination system may be incorporated into an HVAC system. System 100 includes a condenser coil 102, a UVC source 104, UVC reflective material 106, an HVAC blower fan 108, a UVC control module 110, and ductwork 112. Condenser coil 102 and HVAC blower fan 108 may be part of a standard HVAC system into which system 100 is integrated/retrofitted or may be part of a custom HVAC system designed for use with system 100. UVC source 104 may include either or both of a low pressure mercury lamp and/or an LED UVC light. In various aspects, the low pressure mercury lamp and LED UVC light output light in the range of about 250 nm-270 nm, or about 260-265 nm. UVC reflective material 106 lines at least a portion of ductwork 112 and may be, for example, any of galvanized steel, Teflon®, aluminum, brushed aluminum, POREX Virtek®, and sintered PTFE. Other materials that reflect light in the ranges of about 250 nm to about 400 nm may be used.

UVC control module 110 includes a processer and memory and is configured to control, for example, operation of UVC source 104. Control of UVC source 104 includes controlling on/off status, light intensity, duration, strobing, and the like. In some aspects, UVC control module 110 is configured to control operation of HVAC blower fan 108. Control of HVAC blower fan 108 includes controlling on/off status, fan speed, duration, and the like. UVC control module 110 can receive inputs 114 from a variety of sources. Inputs 114 may provide UVC control module 110 with information about, for example, the environment (temperature, humidity, occupancy, HVAC system status, and the like), air flow velocity, and/or various programmed functions. One or more inputs 114 illustrated in FIG. 1 may be sensors that provide data to UVC control module 110. In response to inputs 114, UVC control module 110 may adjust whether or not UVC source 104 is on/off, an intensity of UVC source, and the like in keeping with the various aspects described herein. In some aspects, UVC control module 110 may turn on a low pressure mercury lamp, an LED UVC light, or both.

FIG. 2 is a schematic of a combined active and passive decontamination system 200 according to aspects of the disclosure. System 200 may be incorporated/retrofitted into a standard HVAC system or included in a custom HVAC system designed for use with system 200. System 200 is configured to be coupled to/placed in line with an HVAC vent 202. System 200 may be secured to HVAC vent 202 with various fasteners, clips, and the like. System 200 includes a frame 202 that may be secured to HVAC vent 202 to receive air flowing through HVAC vent 202 to allow system 200 to be easily added to an HVAC system. In some aspects, frame 202 is dimensioned to complement the dimensions of HVAC vent 202 so that system 200 may be more easily placed in line with HVAC vent 202. Frame 202 may include one or more UVC lights 204 positioned on interior walls of frame 202. UVC lights 204 are positioned to direct UVC light toward air passing through frame 202. UVC lights 204 may include either or both of low pressure mercury lamps and LED UVC lights. System 200 includes a UVC control module 206, which may be similar to UVC control module 110 and may perform similar functions. UVC control module 206 receives inputs 212, which are similar to inputs 114. System 200 includes collimator blades 208 that may optionally include a reflective material (similar to UVC reflective material 106) on a surface thereof. Collimator blades 208 permit air to flow from an inside of frame 202 to an outside of frame 202. In some aspects, collimator blades 208 are set at a fixed angle relative to frame 202. In some aspects, collimator blades 208 are attached to frame 202 on pivots or hinges that allow an angle of collimator blades 208 to be adjusted to control a direction and/or velocity of air passing therethrough. In some aspects, system 200 includes a ballast 210 for use with UVC lights 204.

In other embodiments, a uni-strut twist-lock bracket can be provided in connection with the systems described herein. This can be embodied as a bracket designed for holding the horizontal components of the larger in-duct rack system. It may be a simpler bracket than using the standard spring/bolt attachment method to the vertical extrusion, thereby saving time, inventory, and weight for transport. A single bracket can be inserted into the vertical extrusion track, and twisted down to lock in place, which would be a new kind of bracket. The weight of the horizontal bar (or whatever is attached to the bracket) can assist in retaining the twisted position and keeping it locked in place. In addition, the sockets may be mounted onto individual or combined adjustable brackets, which would allow the bulb or bulb angles with the respect to the HVAC duct and the UVC lamp to be customized for each system installation. This arrangement can be used to maximize energy distribution into the HVAC duct “kill chamber” and condenser coils.

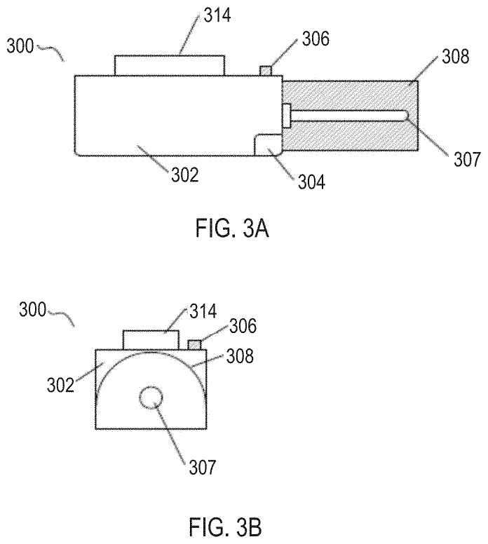

FIGS. 3A and 3B are side and front views, respectively, of an LED UVC wand 300 according to aspects of the disclosure. Wand 300 includes a housing that contains a battery 302, a proximity sensor 304, a spring-loaded safety trigger 306, and a UVC light source 307. UVC light source 307 may be a low pressure mercury lamp or an LED UVC light. In some aspects, wand 300 is battery 302 is a 24V battery to eliminate the need for a transformer component required by some traditional applications. Wand 300 may be fitted with one or more of a shield 308, a timer, and a proximity switch, all of which help limit exposure of UV light to a user of wand 300. Safety trigger 306 is spring-loaded to the off position to give an additional layer of protection to inadvertent exposure. Shield 308 may be lined with reflective material, such as the materials used with UVC reflective material 106. Standard and readily available power tool batteries can drive wand 300 for extended useable life. Wand 300 may also include a handle 314 to assist with handling and carrying wand 300.

In other embodiments, a rack system can be provided which uses support structures such as light bar, ballast heat sync, and wiring chase. UVC light bars are often attached to a separate support frame. Incorporating lamp sockets and ballasts directly onto the support structure frame reduces costs associated with providing separate structures, lamp bars, ballast trays, transportation, and electrical conduit as all components are incorporated into one unit. This can also simplify the installation process. The support structures provide a wiring chase, and the structures can diffuse heat from lamps and ballasts, and they can reduce additional steps in installation as well as material required such as conduit. In certain embodiments, twist-locking and/or friction-fit lamp bar support brackets can be employed.

Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety. Recitation of ranges of values herein is merely intended to serve as a shorthand method of referring individually to each separate value falling within the range. Unless otherwise indicated herein, each individual value is incorporated into the specification as if it were individually recited herein.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa, unless indicated otherwise. In addition, in this application, the use of “or” means “and/or” unless specifically stated otherwise, even though “and/or” may be explicitly used in certain instances.

As used herein, the terms “including,” “containing,” and like terms are understood in the context of this application to be synonymous with “comprising” and are therefore open-ended and do not exclude the presence of additional undescribed or unrecited elements, materials, ingredients or method steps. As used herein, “consisting of” is understood in the context of this application to exclude the presence of any unspecified element, ingredient, or method step. As used herein, “consisting essentially of” is understood in the context of this application to include the specified elements, materials, ingredients or method steps “and those that do not materially affect the basic and novel characteristic(s)” of what is being described.

The examples presented herein are intended to illustrate potential and specific implementations of the present invention. It can be appreciated that the examples are intended primarily for purposes of illustration of the invention for those skilled in the art. No particular aspect or aspects of the examples are necessarily intended to limit the scope of the present invention. All processes and methods described herein can be performed in any suitable order unless otherwise indicated herein or otherwise clearly contradicted by context.

Any element expressed herein as a means for performing a specified function is intended to encompass any way of performing that function including, for example, a combination of elements that performs that function. Furthermore, the invention, as may be defined by such means-plus-function claims, resides in the fact that the functionalities provided by the various recited means are combined and brought together in a manner as defined by the appended claims. Therefore, any means that can provide such functionalities may be considered equivalents to the means shown herein.

It will be appreciated that those skilled in the art will be able to devise various arrangements which, although not explicitly described or shown herein, embody the principles of the present disclosure and are comprised within the scope thereof. Furthermore, all examples and conditional language recited herein are principally intended to aid the reader in understanding the principles described in the present disclosure and the concepts contributed to furthering the art, and are to be construed as being without limitation to such specifically recited examples and conditions. Moreover, all statements herein reciting principles, aspects, and embodiments as well as specific examples thereof, are intended to encompass both structural and functional equivalents thereof. Additionally, it is intended that such equivalents comprise both currently known equivalents and equivalents developed in the future, i.e., any elements developed that perform the same function, regardless of structure. The scope of the present disclosure, therefore, is not intended to be limited to the exemplary aspects and aspects shown and described herein.

Any element expressed herein as a means for performing a specified function is intended to encompass any way of performing that function including, for example, a combination of elements that performs that function. Furthermore, the invention, as may be defined by such means-plus-function claims, resides in the fact that the functionalities provided by the various recited means are combined and brought together in a manner as defined by the appended claims. Therefore, any means that can provide such functionalities may be considered equivalents to the means shown herein.

The processes associated with the present embodiments may be executed by programmable equipment, such as computers. Software or other sets of instructions that may be employed to cause programmable equipment to execute the processes may be stored in any storage device, such as a computer system (non-volatile) memory. Furthermore, some of the processes may be programmed when the computer system is manufactured or via a computer-readable memory storage medium.

It can also be appreciated that certain process aspects described herein may be performed using instructions stored on a computer-readable memory medium or media that direct a computer or computer system to perform process steps. A computer-readable medium may include, for example, memory devices such as diskettes, compact discs of both read-only and read/write varieties, optical disk drives, and hard disk drives. A computer-readable medium may also include memory storage that may be physical, virtual, permanent, temporary, semi-permanent and/or semi-temporary. Memory and/or storage components may be implemented using any computer-readable media capable of storing data such as volatile or non-volatile memory, removable or non-removable memory, erasable or non-erasable memory, writeable or re-writeable memory, and so forth. Examples of computer-readable storage media may include, without limitation, digital video recorders (DVR), RAM, dynamic RAM (DRAM), Double-Data-Rate DRAM (DDRAM), synchronous DRAM (SDRAM), static RAM (SRAM), read-only memory (ROM), programmable ROM (PROM), erasable programmable ROM (EPROM), electrically erasable programmable ROM (EEPROM), flash memory (e.g., NOR or NAND flash memory), content addressable memory (CAM), polymer memory (e.g., ferroelectric polymer memory), phase-change memory, ovonic memory, ferroelectric memory, silicon-oxide-nitride-oxide-silicon (SONOS) memory, magnetic or optical cards, or any other type of media suitable for storing information.

A “computer,” “computer system,” “computing apparatus,” “component,” or “computer processor” may be, for example and without limitation, a processor, microcomputer, minicomputer, server, mainframe, laptop, personal data assistant (PDA), wireless e-mail device, smart phone, mobile phone, electronic tablet, cellular phone, pager, fax machine, scanner, or any other programmable device or computer apparatus configured to transmit, process, and/or receive data. Computer systems and computer-based devices disclosed herein may include memory and/or storage components for storing certain software applications used in obtaining, processing, and communicating information. It can be appreciated that such memory may be internal or external with respect to operation of the disclosed embodiments. In various embodiments, a “host,” “engine,” “loader,” “filter,” “platform,” or “component” may include various computers or computer systems, or may include a reasonable combination of software, firmware, and/or hardware. In certain embodiments, a “module” may include software, firmware, hardware, or any reasonable combination thereof.

In various embodiments of the present invention, a single component may be replaced by multiple components, and multiple components may be replaced by a single component, to perform a given function or functions.

Various embodiments of the systems and methods described herein may employ one or more electronic computer networks to promote communication among different components, transfer data, or to share resources and information. Such computer networks can be classified according to the hardware and software technology that is used to interconnect the devices in the network, such as optical fiber, Ethernet, wireless LAN, HomePNA, power line communication or G.hn. Wireless communications described herein may be conducted with Wi-Fi and Bluetooth enabled networks and devices, among other types of suitable wireless communication protocols. For vehicle systems, networks such as CAN or J1939 may be employed, for example. For V2I (vehicle to infrastructure), or V2X (vehicle to everything) communications, technology such as DSRC or 3GPP may be used, for example. The Controller Area Network (CAN) bus is a serial bus protocol to connect individual systems and sensors as an alternative to conventional multi-wire looms. In certain cases, the CAN bus allows vehicle components to communicate on a single or dual-wire networked data bus. The computer networks may also be embodied as one or more of the following types of networks: local area network (LAN); metropolitan area network (MAN); wide area network (WAN); virtual private network (VPN); storage area network (SAN); or global area network (GAN), among other network varieties.

For example, a WAN computer network may cover a broad area by linking communications across metropolitan, regional, or national boundaries. The network may use routers and/or public communication links. One type of data communication network may cover a relatively broad geographic area (e.g., city-to-city or country-to-country) which uses transmission facilities provided by common carriers, such as telephone service providers. In another example, a GAN computer network may support mobile communications across multiple wireless LANs or satellite networks. In another example, a VPN computer network may include links between nodes carried by open connections or virtual circuits in another network (e.g., the Internet) instead of by physical wires. The link-layer protocols of the VPN can be tunneled through the other network. One VPN application can promote secure communications through the Internet. The VPN can also be used to conduct the traffic of different user communities separately and securely over an underlying network. The VPN may provide users with the virtual experience of accessing the network through an IP address location other than the actual IP address which connects the wireless device to the network. The computer network may be characterized based on functional relationships among the elements or components of the network, such as active networking, client-server, or peer-to-peer functional architecture. The computer network may be classified according to network topology, such as bus network, star network, ring network, mesh network, star-bus network, or hierarchical topology network, for example. The computer network may also be classified based on the method employed for data communication, such as digital and analog networks.

Embodiments of the methods and systems described herein may employ internetworking for connecting two or more distinct electronic computer networks or network segments through a common routing technology. The type of internetwork employed may depend on administration and/or participation in the internetwork. Non-limiting examples of internetworks include intranet, extranet, and Internet. Intranets and extranets may or may not have connections to the Internet. If connected to the Internet, the intranet or extranet may be protected with appropriate authentication technology or other security measures. As applied herein, an intranet can be a group of networks which employ Internet Protocol, web browsers and/or file transfer applications, under common control by an administrative entity. Such an administrative entity could restrict access to the intranet to only authorized users, for example, or another internal network of an organization or commercial entity. As applied herein, an extranet may include a network or internetwork generally limited to a primary organization or entity, but which also has limited connections to the networks of one or more other trusted organizations or entities (e.g., customers of an entity may be given access an intranet of the entity thereby creating an extranet).

Computer networks may include hardware elements to interconnect network nodes, such as network interface cards (NICs) or Ethernet cards, repeaters, bridges, hubs, switches, routers, and other like components. Such elements may be physically wired for communication and/or data connections may be provided with microwave links (e.g., IEEE 802.12) or fiber optics, for example. A network card, network adapter or NIC can be designed to allow computers to communicate over the computer network by providing physical access to a network and an addressing system through the use of MAC addresses, for example. A repeater can be embodied as an electronic device that receives and retransmits a communicated signal at a boosted power level to allow the signal to cover a telecommunication distance with reduced degradation. A network bridge can be configured to connect multiple network segments at the data link layer of a computer network while learning which addresses can be reached through which specific ports of the network. In the network, the bridge may associate a port with an address and then send traffic for that address only to that port. In various embodiments, local bridges may be employed to directly connect local area networks (LANs); remote bridges can be used to create a wide area network (WAN) link between LANs; and/or, wireless bridges can be used to connect LANs and/or to connect remote stations to LANs.

Embodiments of the methods and systems described herein may divide functions between separate CPUs, creating a multiprocessing configuration. For example, multiprocessor and multi-core (multiple CPUs on a single integrated circuit) computer systems with co-processing capabilities may be employed. Also, multitasking may be employed as a computer processing technique to handle simultaneous execution of multiple computer programs.

Although some embodiments may be illustrated and described as comprising functional components, software, engines, and/or modules performing various operations, it can be appreciated that such components or modules may be implemented by one or more hardware components, software components, and/or combination thereof. The functional components, software, engines, and/or modules may be implemented, for example, by logic (e.g., instructions, data, and/or code) to be executed by a logic device (e.g., processor). Such logic may be stored internally or externally to a logic device on one or more types of computer-readable storage media. In other embodiments, the functional components such as software, engines, and/or modules may be implemented by hardware elements that may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Examples of software, engines, and/or modules may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

Additionally, it is to be appreciated that the embodiments described herein illustrate example implementations, and that the functional elements, logical blocks, modules, and circuits elements may be implemented in various other ways which are consistent with the described embodiments. Furthermore, the operations performed by such functional elements, logical blocks, modules, and circuits elements may be combined and/or separated for a given implementation and may be performed by a greater number or fewer number of components or modules. Discrete components and features may be readily separated from or combined with the features of any of the other several aspects without departing from the scope of the present disclosure. Any recited method can be carried out in the order of events recited or in any other order which is logically possible.

Unless specifically stated otherwise, it may be appreciated that terms such as “processing,” “computing,” “calculating,” “determining,” or the like, refer to the action and/or processes of a computer or computing system, or similar electronic computing device, a DSP, ASIC, FPGA or other programmable logic device, discrete gate or transistor logic, discrete hardware components, or any combination thereof designed to perform the functions described herein that manipulates and/or transforms data represented as physical quantities (e.g., electronic) within registers and/or memories into other data similarly represented as physical quantities within the memories, registers or other such information storage, transmission or display devices.

Certain embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, also may mean that two or more elements are not in direct contact with each other, but yet still co-operate or interact with each other. With respect to software elements, for example, the term “coupled” may refer to interfaces, message interfaces, application program interface (API), exchanging messages, and so forth.

It will be appreciated that those skilled in the art will be able to devise various arrangements which, although not explicitly described or shown herein, embody the principles of the present disclosure and are comprised within the scope thereof. Furthermore, all examples and conditional language recited herein are principally intended to aid the reader in understanding the principles described in the present disclosure and the concepts contributed to furthering the art, and are to be construed as being without limitation to such specifically recited examples and conditions. Moreover, all statements herein reciting principles, aspects, and embodiments as well as specific examples thereof, are intended to encompass both structural and functional equivalents thereof. Additionally, it is intended that such equivalents comprise both currently known equivalents and equivalents developed in the future, i.e., any elements developed that perform the same function, regardless of structure. The scope of the present disclosure, therefore, is not intended to be limited to the exemplary aspects and aspects shown and described herein.

The flow charts and methods described herein show the functionality and operation of various implementations. If embodied in software, each block, step, or action may represent a module, segment, or portion of code that comprises program instructions to implement the specified logical functions. The program instructions may be embodied in the form of source code that comprises human-readable statements written in a programming language or machine code that comprises numerical instructions recognizable by a suitable execution system such as a processing component in a computer system. If embodied in hardware, each block may represent a circuit or a number of interconnected circuits to implement the specified logical functions.

Reference to “one embodiment” or “an embodiment” means that a particular feature, structure, or characteristic described in connection with the embodiment is comprised in at least one embodiment. The appearances of the phrase “in one embodiment” or “in one aspect” in the specification are not necessarily all referring to the same embodiment. The terms “a” and “an” and “the” and similar referents used in the context of the present disclosure (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context. The use of any and all examples, or exemplary language (e.g., “such as” or “for example”) provided herein is intended merely to better illuminate the disclosed embodiments and does not pose a limitation on the scope otherwise claimed. No language in the specification should be construed as indicating any non-claimed element essential to the practice of the claimed subject matter. It is further noted that the claims may be drafted to exclude any optional element. As such, this statement is intended to serve as antecedent basis for use of such exclusive terminology as solely, only and the like in connection with the recitation of claim elements, or use of a negative limitation.

Groupings of alternative elements or embodiments disclosed herein are not to be construed as limitations. Each group member may be referred to and claimed individually or in any combination with other members of the group or other elements found herein. It is anticipated that one or more members of a group may be comprised in, or deleted from, a group for reasons of convenience and/or patentability.

While various embodiments of the invention have been described herein, it should be apparent, however, that various modifications, alterations and adaptations to those embodiments may occur to persons skilled in the art with the attainment of some or all of the advantages of the present invention. The disclosed embodiments are therefore intended to include all such modifications, alterations and adaptations without departing from the scope and spirit of the present invention as described herein.

1. A decontamination system comprising:
an HVAC duct;
a UVC light source disposed within the HVAC duct; and
a UVC control module comprising a processor and memory, the UVC control module being in electrical communication with the UVC light source and configured to control operation of the UVC light source.

2. The decontamination system of claim 1, further comprising an HVAC blower fan positioned to direct air through the HVAC duct.

3. The decontamination system of claim 2, wherein the UVC control module is in electrical communication with the HVAC blower fan.

4. The decontamination system of claim 1, wherein the UVC control module is in electrical communication with a sensor that provides data relating to the operation of the decontamination system.

5. The decontamination system of claim 4, wherein the sensor provides the UVC control module with data relating to one or more of a temperature, a humidity, and a number of occupants of an indoor environment associated with the decontamination system.

6. The decontamination system of claim 4, wherein the sensor provides the UVC control module with data relating to an air flow velocity through the HVAC duct.

7. The decontamination system of claim 1, wherein the UVC light source comprise one or both of a low pressure mercury lamp and a LED UVC light.

8. The decontamination system of claim 7, wherein the UVC control module is configured to turn on the LED UVC light independent of the low pressure mercury lamp.

9. The decontamination system of claim 1, wherein the UVC control module is configured to control an intensity and duration of the UVC light source.

10. The decontamination system of claim 1, further comprising a UVC reflective material that covers at least a portion of an interior wall of the HVAC duct, the UVC reflective material chosen from the group consisting of: galvanized steel, Teflon®, aluminum, brushed aluminum, POREX Virtek®, and sintered PTFE.

11. A decontamination system comprising:
a frame configured to be attached to an HVAC vent; and
at least one UVC light secured to an interior of the frame,
wherein at least one wall of the frame comprises a plurality of collimator blades that permit air to pass from an interior of the frame to an exterior of the frame.

12. The decontamination system of claim 11, further comprising a UVC control module comprising a processor and memory, the UVC control module being in electrical communication with the at least one UVC light and configured to control operation of the at least one UVC light.

13. The decontamination system of claim 12, wherein the at least one UVC light comprises one or both of a low pressure mercury lamp and a LED UVC light.

14. The decontamination system of claim 13, wherein the UVC control module is configured to turn on the LED UVC light independent of the low pressure mercury lamp.

15. The decontamination system of claim 12, wherein the UVC control module is configured to control an intensity and duration of the at least one UVC light.

16. The decontamination system of claim 11, wherein the plurality of collimator blades include a UVC reflective material chosen from the group consisting of: galvanized steel, Teflon®, aluminum, brushed aluminum, POREX Virtek®, and sintered PTFE.

17. The decontamination system of claim 11, further comprising a ballast for the UVC light and secured to the frame.

18. The decontamination system of claim 12, wherein the UVC control module is in electrical communication with a sensor that provides data relating to the operation of the decontamination system.

19. The decontamination system of claim 18, wherein the sensor provides the UVC control module with data relating to one or more of a temperature, a humidity, and a number of occupants of an indoor environment associated with the decontamination system.

20. A decontamination wand comprising:
a housing;
a battery disposed within the housing;
a UVC light source selected from the group consisting of a low pressure mercury lamp and a UVC light source;
a spring-loaded safety trigger that is biased in an off position; and
a shield disposed around at least a portion of the UVC light source, the shield comprising a UVC reflective material on an portion of the shield facing the UVC light source.
